SEQUENCE LISTING (1 sequence)

SEQ ID NO: 1 - protein, 13 aa, Artificial Sequence
  Specific Anti-PDPK FA/GSK-3a Antibody
QSTDATPTLTNSS

(19) United States
(12) Patent Application Publication
(10) Pub. No.: US 2012/0309019 A1
(43) Pub. Date: Dec. 6, 2012

(54) DETECTION OF UNHEALTHY BONE MARROW-DERIVED CELL FOR DISEASE PREDISPOSITIONS

(75) Inventor: Shiaw-Der YANG, Hsinchu (TW)

(73) Assignee: NATIONAL TSING HUA UNIVESITY, Hsinchu (TW)

(21) Appl. No.: 13/286,804

(22) Filed: Nov. 1, 2011

Related U.S. Application Data
(63) Continuation-in-part of application No. 12/495,967, filed on Jul. 1, 2009, now abandoned.
(60) Provisional application No. 61/193,703, filed on Dec. 17, 2008.

(51) Int. Cl. G01N 33/573; G01N 33/574

(52) U.S. Cl. 435/7.4

(57) ABSTRACT

Provided herein is a method for detecting the presence of a unique subset of unhealthy bone marrow-derived cell (BMDC) by co-staining the cytoplasmic and/or nuclear expression of PDPK F_{A}/GSK-3α, and specific BMDC markers as the diagnostic indicator for disease predispositions from very early-stage aberrant wound healing, chronic inflammations, immune disorder towards end-stage fibrotic disease and neoplasia in cancer-free human subjects.

### CROSS-REFERENCE TO RELATED APPLICATIONS

This application is a continuation-in-part of U.S. patent application Ser. No. 12/495,967 filed on Jul. 1, 2009, which claims priority upon U.S. Provisional Application No. 61/193,703, filed on Dec. 17, 2008; the contents of which are all herein incorporated by this reference in their entireties. All publications, patents, patent applications, databases and other references cited in this application, all related applications referenced herein, and all references cited therein, are incorporated by reference in their entirety as if restated here in full and as if each individual publication, patent, patent application, database or other reference were specifically and individually indicated to be incorporated by reference.

### FIELD OF THE INVENTION

The present invention relates to a method useful in identifying and detecting a unique subset of bone marrow-derived cell (BMDC) by co-immunostaining the cytoplasmic and/or nuclear protein of proline-directed protein kinase F_{A}/glycogen synthase kinase 3 alpha (PDPK F_{A}/GSK-3α) and a specific subset of BMDC marker protein as the diagnostic indicator for predicting disease predispositions from very early-stage aberrant wound healing, chronic inflammation and immune disorder towards end-stage fibrotic disease and neoplasia in cancer-free human subjects.

### BACKGROUND OF THE INVENTION

It is widely accepted that the bone marrow is a major source of both hematopoietic and mesenchymal stem/progenitor cells and their derivatives recruited and homing to the injured tissue ensuring hematopoietic and skeletal homeostasis. It is now clear that the bone marrow contributes to systemic tissue homeostasis from wound healing to the end-stage fibrotic disease and neoplasia. The intrinsic defect in bone marrow stem/progenitor cells and their derivatives such as stromal and inflammatory cells may represent the major cause for aberrant wound healing, chronic inflammation, systemic diseases and syndromes such as systemic fibroses, infections, obstructions, emboli and multiple organ failures. However, the controversial and paradoxical roles of bone marrow-derived cells (BMDCs) among normal healing wounds, nonhealing wounds such as systemic ulcer and particularly overhealing wounds such as systemic fibrosis and neoplasia remain a great challenge to solve the current bone marrow-related systemic diseases, syndromes and transplantation problems (Bingle et al, J Pathol, 2002, 196:254-265; De Wever & Mareel, J Pathol, 2003, 200:429-447; Dunsmore & Shapiro, J Clin Invest, 2004, 113:180-182; Condeelis & Pollard, Cell, 2006, 124:263-266; Yin & Li, J Clin Invest, 2006, 116:1195-1201; Karnoub et al, Nature, 2007, 449:557-563; Biswas et al, J Immunol, 2008, 180:2011-2017; Kisseleva & Brenner, Exp Biol Med, 2008, 233:109-122; Lin et al, Cells Tissues Organs, 2008, 188:178-188; Schafer & Werner, Nat Rev Mol Cell Biol, 2008, 9:628-638; Valtieri & Sorrentino, J Cell Physiol, 2008, 217:296-300; Wynn, J Pathol, 2008, 214:199-210; Mishra et al, Cancer Res, 2009, 69:1255-1258; Grivennikov et al, Cell, 2010, 140:883-899; Hanahan & Weinberg, Cell, 2011, 144:646-674).

Proline-directed protein kinase F_{A} (PDPK F_{A})/glycogen synthase kinase 3α (GSK-3α) was originally identified as a specific protein phosphatase activating factor A (Vandenheede et al, J Biol Chem, 1980, 255:11768-11774; Yang et al, J Biol Chem, 1980, 255:11759-11767; Woodgett, EMBO J, 1990, 9:2431-2438). In this lab, PDPK F_{A}/GSK-3α was further characterized as a multisubstrate/multifunctional PDPK essential for the development of anti-apoptosis, tumorigenesis, invasion and chemoresistance of various types of cancer cells (Hsu et al, Br J Cancer, 2000, 82:1480-1484; Hsu et al, Cancer, 2000, 89:1004-1011; Yang et al, Clin Cancer Res, 2000, 6:1024-1030; Hsu et al, Cancer, 2001, 92:1753-1758; Hsu et al, Int J Cancer, 2001, 91:650-653; Chung et al, Cancer, 2002, 95:1840-1847; Hsuch et al, Cancer, 2002, 95:775-783; Fu & Yang, Anticancer Res, 2004, 24:1489-1494). However, the systemic role of this signaling molecule particularly in BMDC in normal human subjects remains largely unknown and needs to be established.

### SUMMARY OF THE INVENTION

In one aspect, provided herein is a method for detecting the presence of a cellular expression profile of a bone marrow-derived cell (BMDC) in a cancer-free human subject indicative of a unique subset of unhealthy BMDC, which method comprises determining the expression of PDPK F_{A}/GSK-3α in a specific subset of BMDC in said sample wherein cytoplasmic and/or nuclear protein expression of PDPK F_{A}/GSK-3α in BMDC indicates the presence of a unique subset of unhealthy BMDC for disease predispositions from very early-stage aberrant wound healing, chronic inflammations, immune disorder towards the end-stage fibrotic disease and neoplasia in cancer-free human subjects. In the embodiments, the cytoplasmic and/or nuclear expression of PDPK F_{A}/GSK-3α and BMDC are detected by co-immunostaining technique using antibodies specific to PDPK F_{A}/GSK-3α and specific BMDC surface markers, respectively. The sample can be bone marrow, cord blood, peripheral blood, tissue sample or biopsy.

In another aspect, provided herein is a kit for determining the presence of an unhealthy BMDC in a biological sample comprising at least one reagent for determining the expression of PDPK F_{A}/GSK-3α in a BMDC in said sample, and printed instructions packaged together in a container.

The objects and features of the invention will become more fully apparent when the following detailed description is read in conjunction with the accompanying figures and examples.

### BRIEF DESCRIPTION OF THE DRAWINGS

FIG. 1 depicts the aberrant cytoplasmic and/or nuclear protein expression patterns of PDPK F_{A}/GSK-3α in BMDC by co-immunohistochemical staining of a rare population of CD34⁺ and CD34⁻ stem/progenitor cells associated with very strong cytoplasmic and nuclear expressions (>3+) of PDPK F_{A}/GSK-3α and their possible derivatives with moderate-strong cytoplasmic and/or nuclear protein expression (2+ to 3+) of PDPK F_{A}/GSK-3α in bone marrow of poor outcome leukemia patients. (A). Positive pattern co-staining with CD34, (B). Negative pattern co-staining with CD34.

FIG. 2 depicts the Kaplan-Meier disease-free survival (DFS) and overall survival (OS) curve of various types of patients with very early stage I tumors shown in Table 1 with respect to the presence and absence of the recruited BMDC expressing cytoplasmic and/or nuclear protein expression of PDPK F_{A}/GSK-3α within stroma tissues of breast, bile duct, colorectum, cervix, esophagus, stomach, liver, lung, oral cavity, ovary, pancreas, prostate and kidney. (A). Disease-free survival (DFS), (B). Overall survival (OS).

FIG. 3 depicts the representative cytoplasmic and/or nuclear protein expression pattern of PDPK F_{A}/GSK-3α in poor outcome stage I tumor stromas by co-immunohistochemical staining of a rare population of CD34⁺/vimentin⁻, CD34⁺/vimentin⁺ and CD34⁻/vimentin⁺ cells associated with very strong cytoplasmic and/or nuclear protein expression (>3+) of PDPK F_{A}/GSK-3α and a relatively large population of their derivatives vimentin⁺ fibroblasts/mesenchymal cells, CD68⁺ macrophages and α-smooth muscle actin (α-SMA)⁺ myofibroblast associated with moderate to strong cytoplasmic and/or nuclear protein expression (2+ to 3+) of PDPK F_{A}/GSK-3α. (A). Positive pattern co-staining with CD34, (B). Negative pattern co-staining with CD34, (C). Positive pattern co-staining with vimentin, (D). Negative pattern co-staining with vimentin, (E). Positive pattern co-staining with CD68, (F). Negative pattern co-staining with CD68, (G). Positive pattern co-staining with α-SMA, (H). Negative pattern co-staining with α-SMA.

FIG. 4 establishes PDPK F_{A}/GSK-3α as an early unhealthy signal of a specific unique subset of BMDC by co-immunocytochemical staining of CD34⁺ fibrocytes-like cells associated with strong cytoplasmic and/or nuclear protein expression of PDPK F_{A}/GSK-3α in peripheral blood of children from cancer family or suffering from repeated cycles of inflammations. (A). Positive pattern, (B). Negative pattern.

FIG. 5 further establishes PDPK F_{A}/GSK-3α as a very early unhealthy signal of a specific unique subset of BMDC by co-immunocytochemical staining of CD34⁺ hematopoietic stem/progenitor cells expressing cytoplasmic and/or nuclear PDPK F_{A}/GSK-3α in umbilical cord blood of cancer family child suffering from immune system disorder characteristic of Kawasaki syndrome.

### DETAILED DESCRIPTION OF THE INVENTION

### A. Definition

Unless defined otherwise, all technical and scientific terms used herein have the same meaning as is commonly understood by one of ordinary skill in the art to which this invention belongs. All patents, applications, published applications and other publications, and Genbank Accession numbers referred to herein are incorporated by reference in their entirety. If a definition set forth in this section is contrary to or otherwise inconsistent with a definition set

forth in the patents, applications, published applications and other publications that are herein incorporated by reference, the definition set forth in this section prevails over the definition that is incorporated herein by reference.

As used herein, “a” or “an” means “at least one” or “one or more.”

As used herein, the term “PDPK F_{A}/GSK-3α” refers to the multisubstrate/multifunctional proline-directed protein kinase F_{A} also known as glycogen synthase kinase-3 alpha (Woodgett, EMBO J, 1990, 9:2431-2438; Yang, Curr Cancer Drug Targets, 2004, 4:591-596). The Genbank Accession numbers for this protein are AAD11986 and AAH27984.

As used herein, a unique subset of unhealthy BMDC refers to a specific unique subset of BMDC expressing specific surface marker protein and cytoplasmic and/or nuclear PDPK F_{A}/GSK-3α protein that can be detected within bone marrow of poor outcome leukemia patients or within various tumor stromas of very early stage I cancer patients with poor outcome even after curative resection and regardless of etiological origins as demonstrated in the art. Surprisingly, such type of BMDC can also be detected in cancer families at cancer-free stage as demonstrated in the art. Additionally, based on the well-established evidences and concepts that BMDC can be recruited, accumulated and detected by specific surface markers within tissue stroma and peripheral blood from very early-stage aberrant wound healing process, chronic inflammations and immune disorder towards the end-stage fibrosis and tumorigenesis, this art also disclosed methods for the detection of such type of BMDC, in peripheral blood of children suffering from repeated cycles of inflammations, in umbilical cord blood of child suffering from immune system disorder within three-four years after detection and also in ulcerative tissues. Therefore, a specific subset of BMDC expressing cytoplasmic and/or nuclear PDPK F_{A}/GSK-3α represents a unique subset of unhealthy BMDC associated with disease predispositions and multifaceted cytoplasmic and/or nuclear PDPK F_{A}/GSK-3α signaling molecule represents a new unhealthy signal in a specific unique subset of unhealthy BMDC for disease predispositions from very early-stage aberrant wound healing, chronic inflammation and immune disorder towards the end-stage fibrotic disease and neoplasia in cancer-free human subjects as demonstrated in this art, which has not yet been revealed in any prior arts worldwide.

As used herein, cancer-free human subject refers to human subject without any sign of cancer defined by the current clinic cancer definitions and/or parameters applicable worldwide.

Unless otherwise indicated, all terms used herein have the same meaning as they would to one skilled in the art and the practice of this invention will be employed, conventional techniques of biochemical and clinical pathological technology, which are within the knowledge of those of skill of the art.

### B. Methods and Kits for Detecting Unhealthy BMDC

Any suitable means of detecting aberrant expressions of PDPK FA/GSK-3α in BMDC may be employed. The expression can be determined by assessing protein levels in BMDC from a biological sample. For example, an immunoassay using an antibody specific for PDPK FA/GSK-3α may be employed. Suitable means include, but are not limited to immunohistochemical analysis, immunocytochemical analysis and flow cytometry analysis. With immunohistochemical staining techniques, a cell sample is prepared, typically by dehydration and fixation, followed by reaction with labeled antibodies specific for the gene product coupled, where the labels are usually visually detectable, such as enzymatic labels, florescent labels, luminescent labels, and the like.

According to one embodiment, tissue samples are obtained from subjects and the samples are embedded then cut to e.g. 3-5 μm, fixed, mounted and dried according to conventional tissue mounting techniques. The fixing agent may comprise formalin. The embedding agent for mounting the specimen may comprise, e.g., paraffin. The samples may be stored in this condition. Following deparaffinization and rehydration, the samples are contacted with an immunoreagent comprising an antibody specific for PDPK FA/GSK-3α. The antibody may comprise a polyclonal or monoclonal antibody. The antibody may comprise an intact antibody, or fragments thereof capable of specifically binding PDPK FA/GSK-3α protein. Appropriate polyclonal antisera or other antibody may be prepared by immunizing appropriate host animals with PDPK FA/GSK-3α protein, or a suitable fragment thereof, and collecting and purifying the antisera according to conventional techniques known to those skilled in the art. Monoclonal or polyclonal antibodies specifically reacting with PDPK FA/GSK-3α, may be made by methods well known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York. Also, recombinant antibodies may be produced by methods known in the art, including but not limited to, the methods described in U.S. Pat. No. 4,816,567, or obtained commercially.

The antibody either directly or indirectly bears a suitable detectable label. Alternatively, the detectable label can be attached to a secondary antibody, e.g., goat anti-rabbit IgG, which binds the primary antibody. Frequently, the polypeptides and antibodies are labeled by joining, either covalently or noncovalently, a substance which provides a detectable signal. Suitable labels include, but are not limited to, radionuclides, enzymes, substrates, cofactors, inhibitors, fluorescent agents, chemiluminescent agents, magnetic particles and the like.

Any suitable means can be used to obtain a biological sample from a subject. A biological sample can be bone marrow, blood, tissue, tumor, ascites or pleural effusions.

Kits for monitoring the presence of a cellular expression profile in a subject indicative of unhealthy bone marrow-derived cell (BMDC) for disease predisposition will include at least one container sized to house at least one reagent useful in determining the expression of PDPK FA/GSK-3α in BMDC as defined herein, and printed instructions for assessing whether or not BMDC in a biological sample contain an unhealthy BMDC. As used herein, the term “reagent” means any compound, composition or biological agent (i.e., samples, aliquots or “doses” of cells, antibodies, etc.) useful in carrying out any method provided herein, including but not limited to antibodies for PDPK FA/GSK-3α, buffers and carriers for analysis.

### C. Embodiments

Unless otherwise indicated in the specific embodiments, all immunohistochemical analysis, immunophenotyping analysis, immunocytochemical analysis and statistical analysis followed the below methods.

Production, Identification and Characterization of Specific Anti-PDPK F_{A}/GSK-3α Antibody. The peptide QSTDATPTLTNSS (SEQ ID NO.1), corresponding to the carboxyl terminal region from amino acids 471 to 483 of the sequence of PDPK F_{A}/GSK-3α was synthesized by peptide synthesizer (model 9050, Milligen, Bedford, Mass.). The cysteine residue was added to the NH2 terminus in order to facilitate coupling of the peptide to bovine serum albumin according to the procedure described by Reichlin (1980) using glutaraldehyde as the cross-linker. The antibody production has been through affinity purification and the recognition that could be blocked by the C-terminal peptide from amino acids 471-483 of PDPK F_{A}/GSK-3α to demonstrate the immunospecificity of this anti-PDPK F_{A}/GSK-3α antibody.

Immunohistochemial Analysis. Tissue sections (5 μm) of formalin-fixed, paraffin-embedded tissue containing tumor that showed the maximum extent of tumor cells were dewaxed in xylene and rehydrated in graded concentrations of ethanol. Endogenous peroxidase was blocked with 3% hydrogen peroxide followed by bovine serum albumin blocking for 5 minutes. The slides were next incubated with anti-PDPK F_{A}/GSK-3α antibody (2 μg/mL) diluted in 0.05 M Tris buffer, pH 7.4, at 4° C. for 16 hours followed by 20-minute incubation at room temperature with super enhancer (Super Sensitive™ Non-Biotin Detection System, [BioGenex, San Ramon, Calif.]), and another 30-minute incubation with polymer-HRP (Super Sensitive™) label. Immunostaining was finally developed with DAB (3-3′ diaminobenzidine tetrahydrochloride). After quenching the enzyme reaction, slides were incubated in DS-enhancer (Zymed, San Francisco, Calif.) at room temperature for five minutes to prevent the interaction between two staining system. Then, slides were incubated with CD34, vimentin, CD68 or α-SMA antibody for one hour at room temperature. After washing, slides were incubated with anti-mouse alkaline phosphatase for 30 minutes at room temperature. BCIP/NBT solution was used for visualization of the bound antibody. Sections were counterstained with methyl green solution. All the BMDC specific antibodies are commercially-available.

Immunocytochemical analysis. On average 1×10⁶ cells will be cytocentrifuged onto polylysine-coated slides at 700 rpm for 3 minutes at room temperature (Kubota 5200, Japan). Before staining, the cytospots will be fixed with 3.7% paraformaldehyde for 15 minutes and treated with 0.2% triton X-100 for 90 seconds. Endogenous peroxidase will be blocked with 3% hydrogen peroxide followed by bovine serum albumin blocking for 10 minutes. The slides will be incubated with anti-PDPK F_{A}/GSK-3α antibody (2 μg/mL) diluted in 0.05 M Tris buffer, pH 7.4, at 4° C. for 16 hours followed by 20-minute incubation at room temperature with super enhancer (Super Sensitive™ Non-Biotin Detection System, [BioGenex, San Ramon, Calif.]), and another 30-minute incubation with polymer-HRP (Super Sensitive™) label. Immunostaining was finally developed with DAB (3-3′ diaminobenzidine tetrahydrochloride). After quenching the enzyme reaction, slides were incubated in DS-enhancer (Zymed, San Francisco, Calif.) at room temperature for five minutes to prevent the interaction between two staining system. Then, slides were incubated with CD34, vimentin, CD68 or α-SMA antibody for one hour at room temperature. After washing, slides were incubated with anti-mouse alkaline phosphatase for 30 minutes at room temperature. BCIP/NBT solution was used for visualization of the bound antibody. Sections were counterstained with methyl green solution. All the specific BMDC marker proteins antibodies are commercially-available

Statistical analysis. In the statistical analyses, the samples were dichotomized as with versus without the presence of a specific subset of BMDC expressing cytoplasmic and/or nuclear PDPK F_{A}/GSK-3α. Overall survival was calculated from the date of diagnosis to the date of death or last follow-up. Disease-free survival was measured from the date of diagnosis to the date of recurrence, metastasis, death or last follow-up. The Kaplan-Meier method was used to determine the survival probability, and the log-rank test was used to compare the survival curves between groups. Two groups were compared by using chi-square, student's t and Mann-Whitney U tests, whichever appropriate. The impact of the presence of recruited BMDC expressing cytoplasmic and/or nuclear PDPK F_{A}/GSK-3α was analyzed by the Cox proportional hazards regression model. Logistic regression was used to calculate the risk of the presence of a unique subset of BMDC expressing cytoplasmic and/or nuclear PDPK F_{A}/GSK-3α in cancer family at cancer-free stage. P<0.05 was considered statistically significant.

The study was performed under the approved research projects and grants from National Science Council in Taiwan and approved by the informed consents and the Institution's Surveillance and Ethics Committee.

The following examples are offered to illustrate the invention.

### Example I

### Aberrant Cytoplasmic and/or Nuclear Protein Expression of PDPK F_{A}/GSK-3α in a Unique Subset of BMDC in Poor Outcome Leukemia Patients

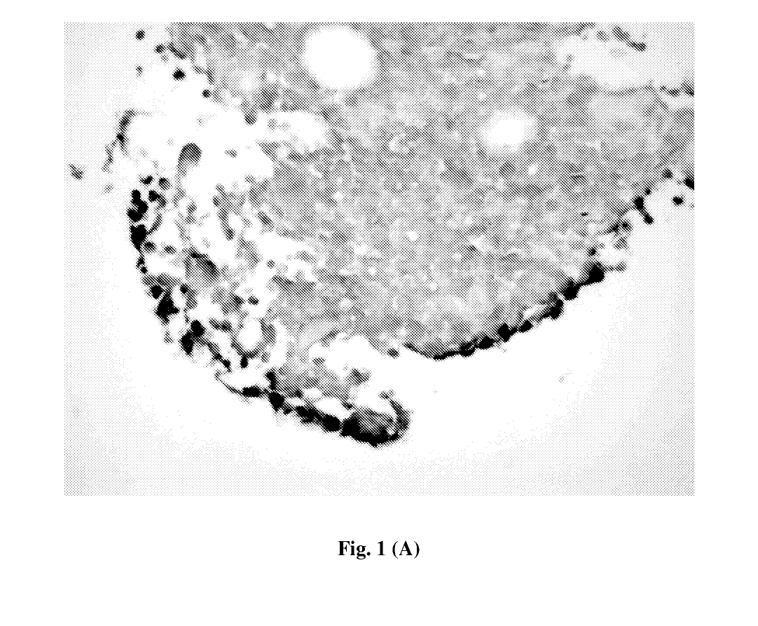

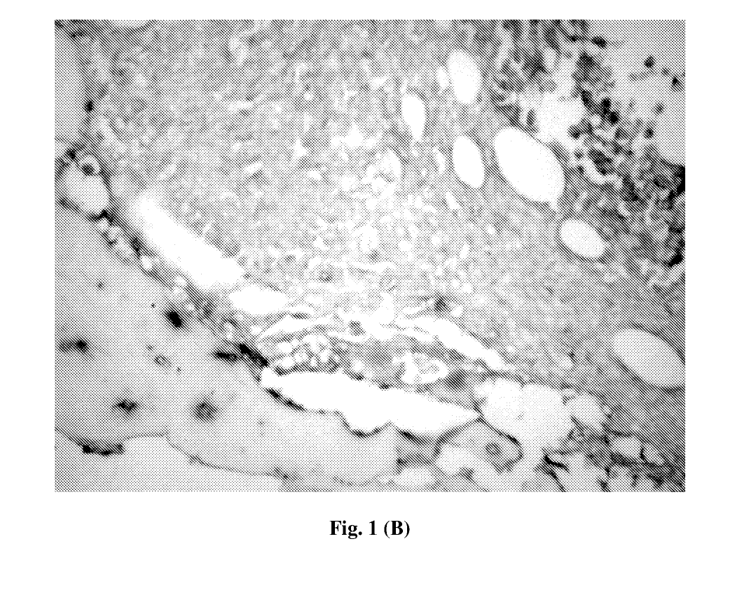

To establish the systemic role of PDPK F_{A}/GSK-3α in BMDC, an independent cohort study on bone marrow of 24 leukemia patients was performed. Aberrant cytoplasmic and/or nuclear protein expression of PDPK F_{A}/GSK-3α could be frequently detected in bone marrow of leukemia patients with progressive diseases. In a cohort study of 24 cases, 14 cases were negative and 10 cases were found to be associated with aberrant cytoplasmic and/or nuclear expressions of PDPK F_{A}/GSK-3α. The immunophenotyping analysis with CD34 further revealed that a rare population of CD34⁺ hematopoietic stem/progenitor cells, and CD34⁻ mesenchymal stem/progenitor cells (Moioli et al, PLoS ONE, 2008, 3:e3922) associated with very strong cytoplasmic and/or nuclear protein expression (>3+) of PDPK F_{A}/GSK-3α and their derivatives associated with moderate to strong cytoplasmic and/or nuclear protein expression (2+ to 3+) of PDPK F_{A}/GSK-3α (FIG. 1A) in contrast to the negative cases (FIG. 1B) could be frequently detected in bone marrow of those patients with progressive diseases. The results from bone marrow study provide initial evidence to demonstrate a specific unique subset of BMDC expressing cytoplasmic and/or nuclear PDPK F_{A}/GSK-3α in poor outcome leukemia patients.

### Example II

### Aberrant Cytoplasmic and/or Nuclear Protein Expression of PDPK F_{A}/GSK-3α in a Unique Subset of Recruited BMDC within the Stromas of Various Types of Poor Outcome Stage I Tumors

In this embodiment, the stroma tissue specimens used for immunohistochemical analysis were obtained through a detailed retrospective review of the medical records of 367 patients with very early stage I tumors who had treatments at National Taiwan University Hospital, Taipei, Taiwan, between 1987 and 2004. Table 1 shows the stroma tissue locations, age, gender, status of survival and PDPK F_{A}/GSK-3α. Patients were observed until April, 2006.

TABLE 1
Patients Characteristics

| Characteristics | | Case number | Percentage (%) |
|---|---|---|---|
| Origin of stroma tissue | Breast | 17 | 4.6 |
| location | Bile duct | 58 | 15.8 |
| | Colorectum | 9 | 2.5 |
| | Cervix | 18 | 4.9 |
| | Esophagus | 7 | 1.9 |
| | Stomach | 45 | 12.3 |
| | Liver | 14 | 3.8 |
| | Lung | 69 | 18.8 |
| | Oral cavity | 3 | 0.8 |
| | Ovary | 30 | 8.2 |
| | Pancreas | 42 | 11.4 |
| | Prostate | 8 | 2.2 |
| | Kidney | 47 | 12.8 |
| Gender | Male | 180 | |
| | Female | 187 | |
| Age | Range (years) | 21-89 | |
| | Mean ± SD | 58.8 ± 12.7 | |
| Status | Alive | 270 | 73.6 |
| | Death | 97 | 26.4 |
| PDPK F_{A}/GSK-3α status | Negative | 240 | 65.4 |
| | Positive | 127 | 34.6 |

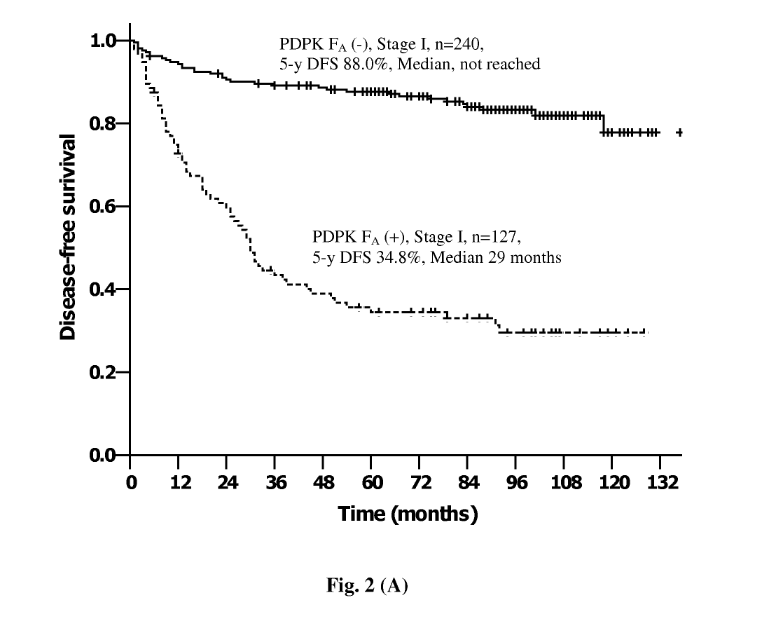

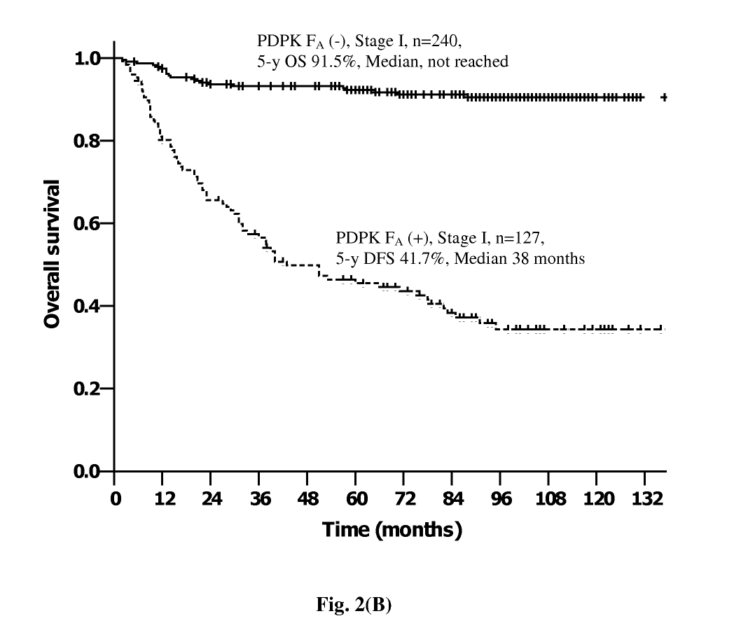

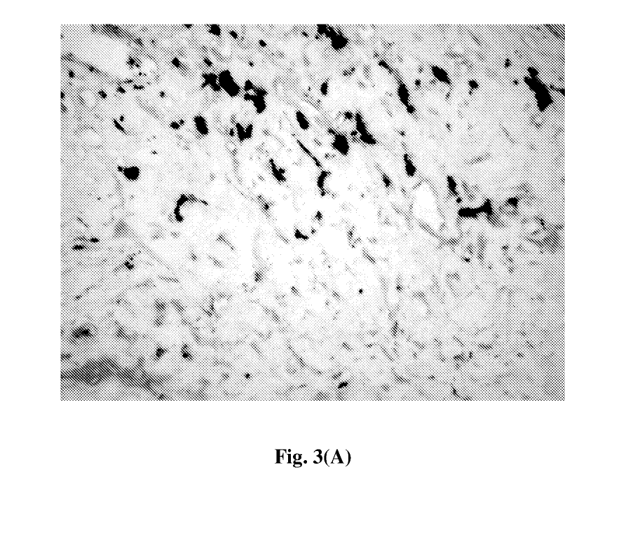

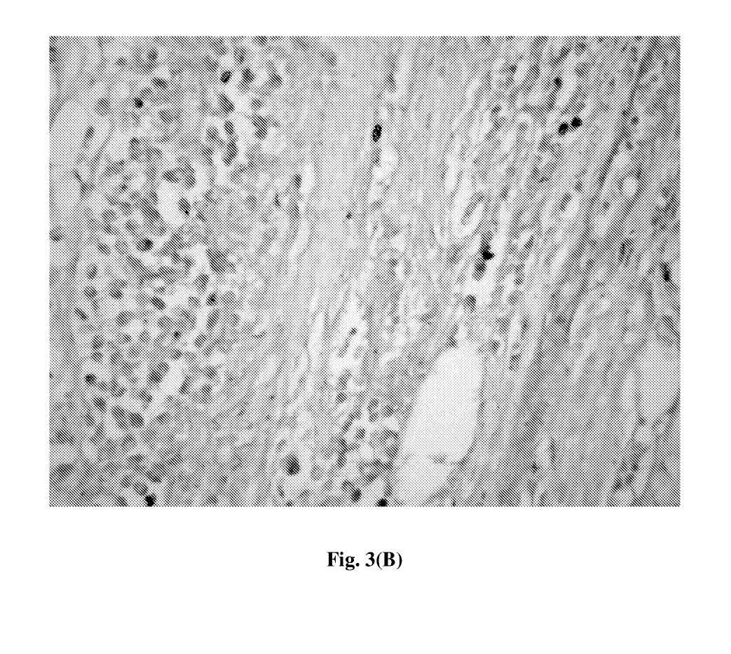

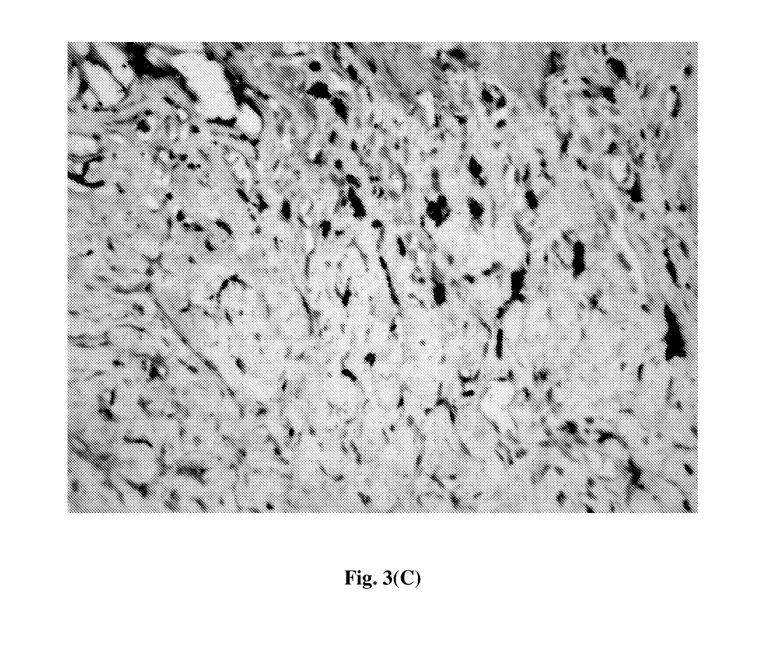

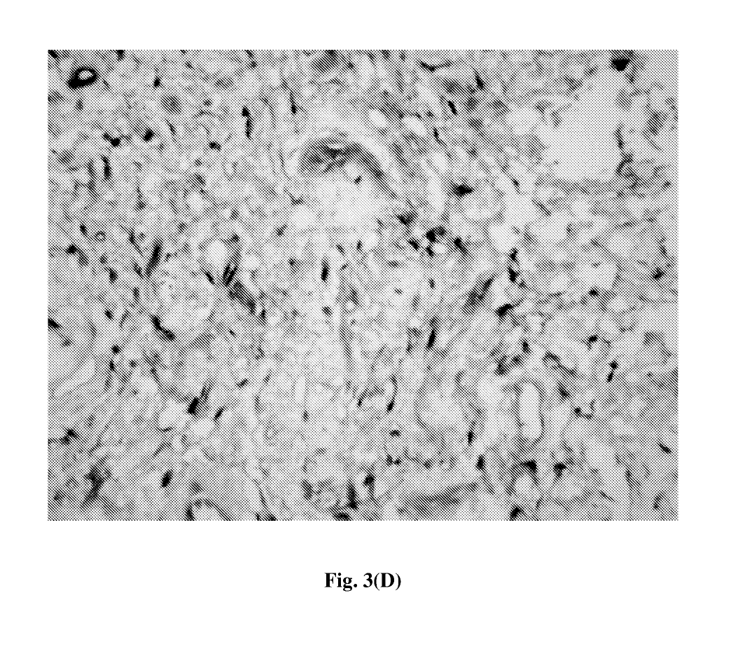

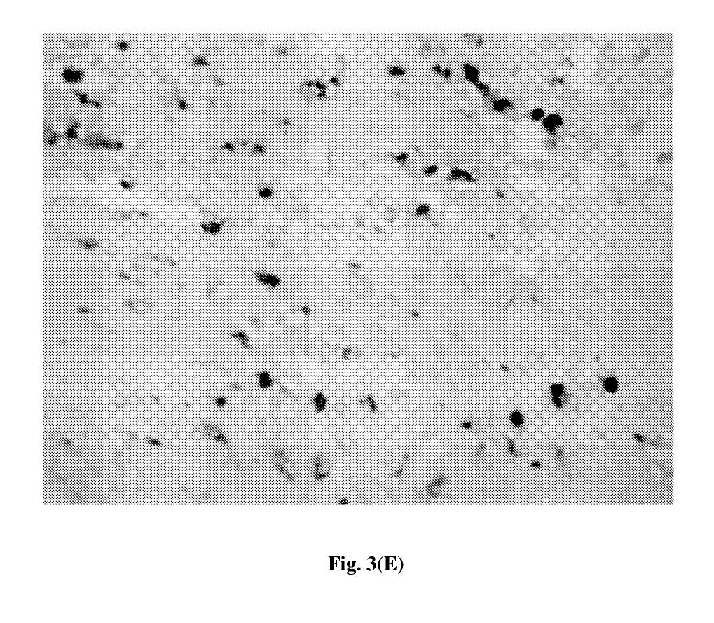

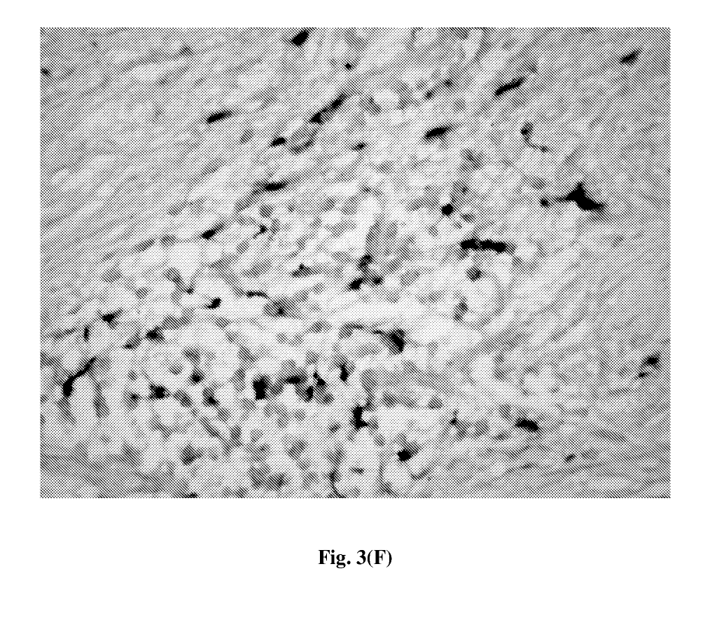

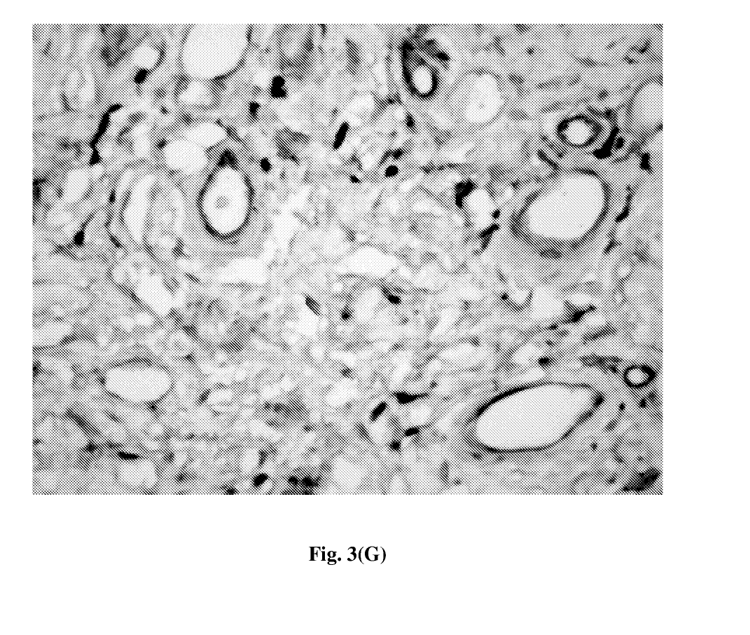

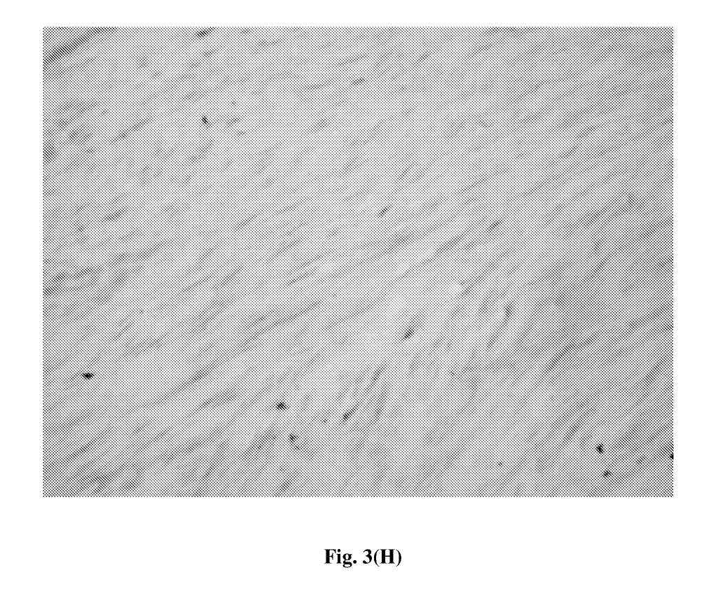

Although Hanahan and Weinberg (Cell, 2000, 100:57-70) had enumerated the hallmarks of cancer, the developed therapies still failed approximately half populations of the patients. The recent studies have challenged that the incurable early-stage tumors are merely the end products of the systemic bone-marrow diseases and represent only a small population of systemic chronic diseases. The intimate relationship between bone marrow and tumor stroma is now more widely acknowledged. There is increasing evidence that bone marrow contributes to stromal lineages in a variety of situations from aberrant wound healing to end-stage systemic fibrotic diseases and/or neoplasia and BMDC is a cell type that is recruited in large numbers to the stroma of fibrotic tissues and/or developing tumors (Direkze & Alison, Hematol Oncol, 2006, 24:189-195; Bellini & Mattoli, Lab Invest, 2007, 87:858-870; Karnoub et al, Nature, 2007, 449:557-563; Lin et al, Cells Tissues Organs, 2008, 188:178-188). In agreement with this notion, a unique subset of recruited BMDC expressing cytoplasmic and/or nuclear PDPK F_{A}/GSK-3α could be frequently detected within the stromas of various types of stage I tumors with poor prognosis even after curative resections. In a cohort study of 367 stage I tumors, 240 cases were negative and 127 cases were associated with aberrant cytoplasmic and/or nuclear expression of PDPK F_{A}/GSK-3α. Patients if associated with aberrant cytoplasmic and/or nuclear expression of this signaling molecule within tumor stromas tend to develop poor outcome (FIG. 2). Immunophenotyping analysis with CD34, CD68, vimentin and α-SMA further revealed that a rare population of CD34⁺/vimentin⁺ hematopoietic stem/progenitor cells, CD34⁺/vimentin⁺ fibrocytes and CD34⁻/vimentin⁺ mesenchymal stem/progenitor cells (Bucala, Fibrocytes, World Scientific Pub Co Inc, 2007:1-18; Moioli et al, PLoS ONE, 2008, 3:e3922) associated with very strong cytoplasmic and/or nuclear protein expression (>3+) of PDPK F_{A}/GSK-3α (FIGS. 3A and C) essentially as described in Example I along with a relatively large population of CD68⁺ macrophages (FIG. 3E), vimentin⁺ fibroblasts/mesenchymal cells (FIG. 3C) and α-SMA⁺ myofibroblasts (FIG. 3G) associated with moderate to strong cytoplasmic and/or nuclear protein expression (2+ to 3+) of PDPK F_{A}/GSK-3α in contrast to the negative cases with good outcome (see FIGS. 3B, D, F and H) could be detected predominantly within the stromas of incurable stage I tumors with poor prognosis even after curative resections. Regardless of the etiology of the stromas of breast, bile duct, colorectum, cervix, esophagus, stomach, liver, lung, oral cavity, ovary, pancreas, prostate and kidney, if associated with aberrant cytoplasmic and/or nuclear expression of PDPK F_{A}/GSK-3α, the stage I patients tend to develop systemic fibroses, infections, obstructions, emboli, ulcer, UGI bleeding and multiple organ failures. The Cox regression analysis further revealed that the positive stage I patients had more than 6-fold the risk of developing systemic diseases and syndromes compared to the negative stage I patients (hazard ratio 6.258, 95% CI 4.125-9.493 for disease-free survival, P<0.001). Thus, the disease entities of stage I tumors if incurable with local excisions, are no longer tumor-specific diseases; they are host-specific, bone marrow-specific diseases at the onset. Taken together, the results provide further evidences to demonstrate a unique subset of BMDC expressing cytoplasmic and/or nuclear PDPK F_{A}/GSK-3α in poor outcome stage I tumors. The combined results support the current notion that the stromas of developing tumors are often accompanied by the recruitment of a variety of BMDC which entails the constant deposition of growth factors, cytokines, matrix-remodeling protein and connective tissue elements to cause multiple organ failures (Hanahan & Weinberg, Cell, 2011, 144:646-674).

### Example III

### Aberrant Cytoplasmic and/or Nuclear Protein Expression of PDPK F_{A}/GSK-3α in a Specific Unique Subset of Recruited BMDC within Inflammatory Fibrotic Tissues

It is now clear that BMDC can be recruited and homing to the injured tissues throughout a large variety of vital organs from very early-stage aberrant wound healing process and chronic inflammations towards the end-stage fibrotic disease and neoplasia. The aberrant wound healing represents a significant cause of morbidity and mortality for a large portion of the population. Thus, on the bright side, BMDCs are a potential therapy for many diseases including end-stage ischemic heart disease, arterial stenosis and osteogenesis imperfecta. Conversely, on the dark side, BMDCs contribute to aberrant wound healing, chronic inflammations and systemic fibrosis in many vital organs including liver, lung, kidney, intestine and bone marrow as well as to systemic fibrosis surrounding various types of tumors (Le Bousse-Kerdiles et al, Eur Cytokine Netw, 2008, 19:69-80; Lin et al, Cells Tissues Organs, 2008, 188:178-188). Due to multisubstrate/multifunctional PDPK nature, the aberrant cytoplasmic and/or nuclear expression of PDPK F_{A}/GSK-3α in BMDC may represent the underlying mechanism for disease predispositions from very early-stage aberrant wound healing, chronic inflammation and immune disorder towards the end-stage fibrotic disease and neoplasia. In accordance with this notion, the aberrant cytoplasmic and/or nuclear expressions of PDPK F_{A}/GSK-3α in a specific unique subset of recruited BMDC indeed could be frequently detected in inflammatory fibrotic tissues. In an independent cohort study of 35 squamous hyperplastic tissues, comprising 26 cases without fibrosis and 9 cases with fibrosis, aberrant cytoplasmic and/or nuclear protein expression of PDPK F_{A}/GSK-3α in BMDC as shown in FIGS. 1 and 3 were detected predominantly in hyperplastic tissues with fibrosis; 7 of 9 were found to be positive with PDPK F_{A}/GSK-3α. Conversely, 18 of 26 cases of fibrosis-negative tissues were found to be PDPK F_{A}/GSK-3α-negative (Chi-square test, P=0.022). Immunophenotyping analysis with CD34, CD68, vimentin and α-SMA further revealed that a rare population of CD34⁺/vimentin⁻ hematopoietic stem/progenitor cells, CD34⁺/vimentin⁺ fibrocytes and CD34⁻/vimentin⁺ mesenchymal stem/progenitor cells associated with very strong cytoplasmic and/or nuclear protein expression (>3+) of PDPK F_{A}/GSK-3α along with a large population of CD68⁺ macrophages, vimentin⁺ fibroblasts and mesenchymal cells and α-SMA⁺ myofibroblasts associated with moderate to strong cytoplasmic and/or nuclear protein expression (2+ to 3+) of PDPK F_{A}/GSK-3α could be frequently detected in inflammatory fibrotic tissues in contrast to the negative cases without fibrosis essentially described in Examples I and II (not further illustrated). There is increasing evidence that BMDCs as shown in FIG. 1 contribute to the new population of stem/progenitor cells and derivatives at the tissue site particularly during aberrant wound healing in inflammatory fibrotic tissues. Fibrotic diseases are an important cause of morbidity and mortality worldwide. They occur in a large variety of vital organs and the process of systemic fibrosis is common to all tissues. The different BMDC types involved in this process may work together to stimulate the deposition of connective tissue elements that progressively destroy the normal tissue architecture with the loss of organ functions leading to death (Dunsmore & Shapiro, J Clin Invest, 2004, 113:180-182; Bellini & Mattoli, Lab Invest, 2007, 87:858-870; Bucala, Fibrocytes, World Scientific Pub Co Inc, 2007:1-18; Le Bousse-Kerdiles et al, Eur Cytokine Netw, 2008, 19:69-80; Lin et al, Cells Tissues Organs, 2008, 188:178-188). Taken together, the results provide evidence to demonstrate a unique subset of BMDC expressing aberrant cytoplasmic and/or nuclear PDPK F_{A}/GSK-3α associated with fibrosis characteristic of aberrant wound healing and chronic inflammations as described above. All the results as shown in Examples I-III taken together provide further evidence to demonstrate a unique subset of BMDC expressing cytoplasmic and/or nuclear PDPK F_{A}/GSK-3α in poor outcome leukemia, poor outcome stage I cancer as well as in inflammatory fibrotic tissues, which provides methods and compositions for predicting disease predispositions from very early-stage inflammations towards the end-stage fibrotic disease and neoplasia.

### Example IV

### Aberrant Cytoplasmic and/or Nuclear Protein Expression of PDPK F_{A}/GSK-3α in a Unique Subset of Recruited/Circulating BMDC in Peripheral Blood of Children from Cancer Family or Suffering from Repeated Cycles of Inflammations

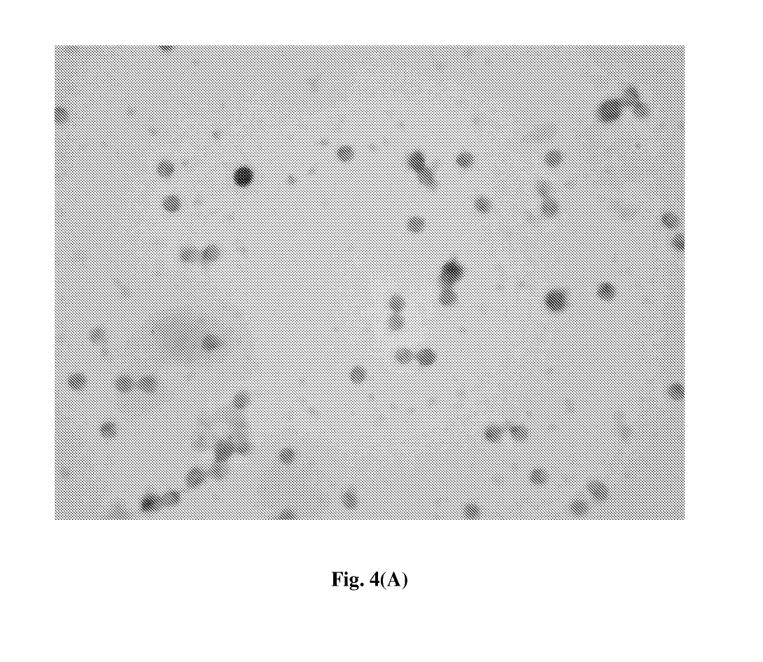

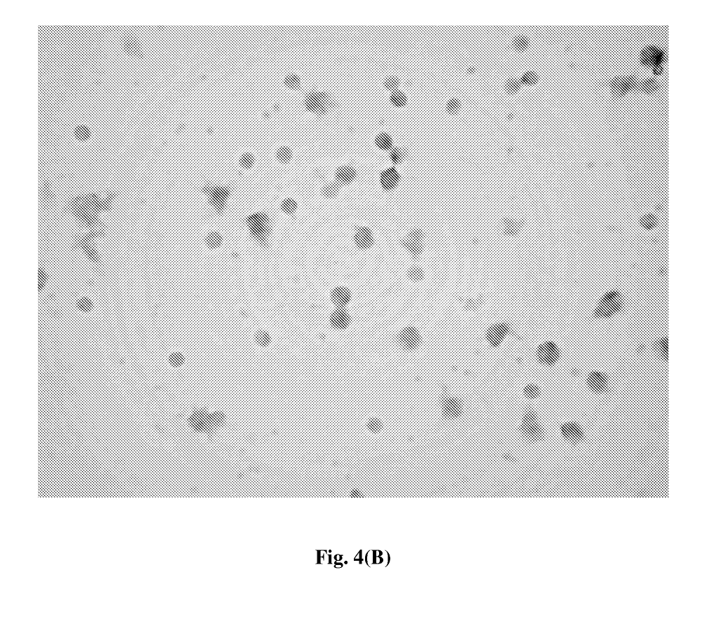

There is increasing evidence that CD34⁺/vimentin⁺ fibrocytes, the bone marrow-derived mesenchymal progenitor in peripheral blood contribute to the new population of fibroblasts and myofibroblasts during aberrant wound healing and inflammatory fibrotic processes (Bucala et al, Mol Med, 1994, 1:71-81; Schmidt et al, J Immunol, 2003, 171:380-389; Bellini & Mattoli, Lab Invest, 2007, 87:858-870; Bucala, Fibrocytes, World Scientific Pub Co Inc, 2007:1-18; Lin et al, Cells Tissues Organs, 2008, 188:178-188). In agreement with this notion, CD34⁺ fibrocyte-like cell associated with strong cytoplasmic and/or nuclear protein expression of PDPK F_{A}/GSK-3α could be detected in peripheral blood of all the tested 3 children from 3 different cancer families suffering from repeated cycles of inflammation within 3-4 years after detection of such type of unhealthy BMDC (FIG. 4). Such type of BMDC was also associated with human hypertrophic scars, nephrogenic systemic fibrosis, atherosclerotic lesions and pulmonary fibrosis. Taken together, the results provide comprehensive evidences to demonstrate a specific unique subset of BMDC expressing cytoplasmic and/or nuclear PDPK F_{A}/GSK-3α for disease predispositions from end-stage poor outcome leukemia, poor outcome stage I tumors and inflammatory fibrotic tissues to the very early-stage aberrant wound healing/chronic inflammation/immune disorder in cancer family children at the cancer-free stage.

It is now well-recognized that BMDCs represent the original resource contributing to tissue homeostasis from very early-stage aberrant wound healing towards the end-stage systemic fibrotic disease and/or neoplasia. The intrinsic defect in bone marrow stem/progenitor cells and derivatives such as stromal/mesenchymal and myeloid/immune/inflammatory cells may represent the major cause for disease predispositions from very early-stage aberrant wound healing/chronic inflammation towards the end-stage fibrosis and neoplasia (Grivennikov et al, Cell, 2010, 140:883-899; Hanahan & Weinberg, Cell, 2011, 144:646-674). Research and studies have found that certain gene abnormality increase the chances of a person to develop certain kinds of cancers, depending on family history. In accordance with this concept, of 33 normal individuals without any sign of cancer defined by the current clinic cancer definitions (parameters) applicable worldwide studied (13 male and 20 female) with ages ranging from 10 to 65 years (mean±SD, 42.5±13.1), the cancer family members tend to have unhealthy BMDC detected in their blood (P=0.001). The logistic regression revealed that cancer family members had more than 13-fold the risk of the presence of unhealthy BMDC compared to non-cancer family people (OR for the presence of unhealthy BMDC 13.333, 95% CI 2.454-72.452, P=0.003; Table 2). The results taken together further demonstrated that the cancer-free cancer family members tend to have higher risk of developing a specific unique type of BMDCs expressing cytoplasmic and/or nuclear PDPK F_{A}/GSK-3α for disease predispositions from very early-stage aberrant wound healing, chronic inflammation and immune disorder toward the end-stage fibrotic disease and neoplasia.

TABLE 2
The risk of the presence of unhealthy BMDC in cancer families

| | Correlation^{†} | | | | | |
|---|---|---|---|---|---|---|
| | The | | | Logistic regression^{‡} | | |
| | presence | | | OR for | | |
| | of unhealthy | | | the presence | | |
| | cell | | | of unhealthy | | |
| | − | + | P* | BMDC | 95% CI | P* |
| Age | 44.4 ± 13.4 | 41.1 ± 13.1 | NS | | | |
| Gender | | | | | | |
| Male | 5 | 8 | | | | |
| Female | 9 | 11 | NS | | | |
| Group | | | | | | |
| Non-cancer family | 10 | 3 | | 1 | | |
| Cancer family | 4 | 16 | 0.001 | 13.333 | 2.454-72.452 | 0.003 |

Abbreviations:
OR, odds ratio;
95% CI, 95% confidence interval
*P < 0.05 was considered statistically significant (^{†}chi-square test and ^{‡}logistic regression)

### Example V

### Aberrant Cytoplasmic and/or Nuclear Protein Expression of PDPK F_{A}/GSK-3α in a Unique Subset of BMDC in Umbilical Cord Blood of Cancer Family Child Suffering from Kawasaki Syndrome

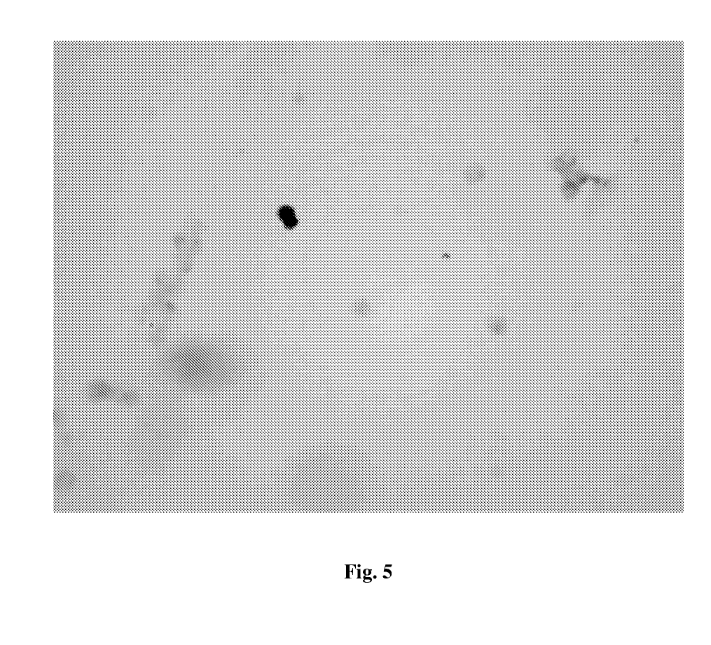

It is now widely accepted that the intrinsic defect in bone marrow stem/progenitor cells and their derivatives such as stromal/mesenchymal and myeloid/immune/inflammatory cells may represent the major cause for very early-stage aberrant wound healing/chronic inflammation/immune system disorder towards the end-stage fibrotic disease and neoplasia (Grivennikov et al, Cell, 2010, 140:883-899). In agreement with the results presented above, a unique subset of BMDC expressing cytoplasmic and/or nuclear PDPK F_{A}/GSK-3α could also be detected in the umbilical cord blood of cancer family children suffering from immune system disorder characteristic of Kawasaki syndrome within 3-4 years after detection (FIG. 5). The results provide further evidence to demonstrate the systemic role of PDPK F_{A}/GSK-3α as a very early unhealthy signaling molecule associated with cord blood and bone marrow stem/progenitor cells. All the results combined demonstrated a unique subset of BMDC expressing cytoplasmic and/or nuclear PDPK F_{A}/GSK-3α throughout the life span of a subject for disease predispositions from a very early symptom-developing stage towards a very final syndrome-developed stage. Thus, the invention provides methods and compositions for predicting disease predispositions from very early-stage aberrant wound healing, chronic inflammation and immune disorder toward the end stage fibrotic disease and neoplasia in cancer-free human being for personalized healthcare.

### Example VI

### Aberrant Cytoplasmic and/or Nuclear Protein Expression of PDPK F_{A}/GSK-3α in a Specific Unique Subset of Recruited BMDC within Aberrant Wound Healing Ulcerative Tissue

It is now well-recognized that BMDCs represent the original resource contributing to tissue homeostasis from very early-stage aberrant wound healing towards the end-stage systemic fibrotic disease and/or neoplasia. The intrinsic defect in bone marrow stem/progenitor cells and derivatives such as stromal/mesenchymal and myeloid/immune/inflammatory cells may represent the major cause for disease predispositions for very early-stage aberrant wound healing/chronic inflammation/immune system disorder towards the end-stage fibrotic disease and neoplasia (Grivennikov et al, Cell, 2010, 140:883-899; Hanahan & Weinberg, Cell, 2011, 144:646-674). In agreement with this notion, a unique subset of BMDC expressing cytoplasmic and/or nuclear PDPK F_{A}/GSK-3α as described above could also be detected within aberrant wound healing ulcerative tissue stromas. In an independent cohort study of 39 oral tissues with ulcer, 34 of 39 cases were associated with aberrant cytoplasmic and/or nuclear protein expression of PDPK F_{A}/GSK-3α in a hierarchical population of BMDCs comprising a rare population of CD34⁺/vimentin⁻ hematopoietic stem/progenitor cells and CD34⁻/vimentin⁺ mesenchymal stem/progenitor cells associated with very strong cytoplasmic and/or nuclear protein expressions (>3+) of PDPK F_{A}/GSK-3α and their derivatives associated with moderate to strong cytoplasmic and/or nuclear protein expression (2+ to 3+) of PDPK F_{A}/GSK-3α within tissue stroma essentially as described above (not further illustrated). Taken together, this invention demonstrated a unique subset of BMDC expressing cytoplasmic and/or nuclear PDPK F_{A}/GSK-3α as a very early diagnosticator for predicting disease predispositions from very early-stage aberrant wound healing/chronic inflammation/immune disorder towards the end-stage fibrotic disease and neoplasia in cancer-free human subjects for personalized healthcare.

In summary, the present invention provides a detection system to diagnose and predict the disease status of bone marrow and cord blood prior to transplantations to decrease risk of transmission of genetic disease. The specific unique subset of unhealthy BMDC associated with cytoplasmic and/or nuclear protein expression of PDPK F_{A}/GSK-3α presented in this invention, thus provide method and composition for detecting and predicting disease predispositions from very-early-stage aberrant wound healing, chronic inflammation and immune disorder toward the end-stage fibrotic disease and neoplasia in cancer-free human subject for personalized healthcare. In conclusion, by definition, the specific subset of BMDC expressing cytoplasmic and/or nuclear PDPK F_{A}/GSK-3α refers to a unique subset of BMDC that can be detected within bone marrow of poor outcome leukemia patients or within various stroma tissues of very early stage I cancer patients with poor outcome even after curative resection and regardless of etiological origins as demonstrated in the art (Please check Example I and II, Table I and FIGS. 1-3). Surprisingly, such type of BMDC can also be detected in cancer-free cancer families at higher risk of developing disease predispositions as demonstrated in the art (Please check Table II). Additionally, based on the well-established evidences and concepts that BMDC can be recruited, accumulated and detected by specific surface markers within tissue stroma and peripheral blood from very early-stage aberrant wound healing process, chronic inflammations and immune disorder towards the end-stage fibrotic disease and neoplasia, this art also disclosed methods and compositions for the detection of such type of BMDC, in peripheral blood of children suffering from repeated cycles of inflammations (Please check Example IV), in umbilical cord blood of child suffering from immune system disorder within three-four years after detection (Please check Example V) and also in aberrant wound healing ulcerative tissues (Please check Example VI). Therefore, a specific subset of BMDC expressing cytoplasmic and/or nuclear PDPK F_{A}/GSK-3α represents a unique subset of unhealthy BMDC associated with disease predispositions and multifaceted cytoplasmic and/or nuclear PDPK F_{A}/GSK-3α signaling molecule represents a new unhealthy signal in a specific unique subset of unhealthy BMDC for predicting disease predispositions as demonstrated in this art. The unique technique in this art is to co-stain both a specific subset of BMDC marker protein and signaling molecule PDPK F_{A}/GSK-3α protein to detect and differentiate a specific unique subset of abnormal unhealthy BMDC from normal healthy BMDC for predicting disease predispositions from very early-stage aberrant wound healing/chronic inflammation/immune disorder towards end-stage fibrotic disease and neoplasia in cancer-free human beings for personalized healthcare, which has not yet been revealed in any prior arts worldwide.

### REFERENCES

Bellini A, Mattoli S (2007) The role of the fibrocyte, a bone marrow-derived mesenchymal progenitor, in reactive and reparative fibroses. Lab Invest 87: 858-870
Bingle L, Brown N J, Lewis C E (2002) The role of tumour-associated macrophages in tumour progression: implications for new anticancer therapies. J Pathol 196: 254-265
Biswas S K, Sica A, Lewis C E (2008) Plasticity of macrophage function during tumor progression: regulation by distinct molecular mechanisms. J Immunol 180: 2011-2017
Bucala R (2007) Fibrocytes. Fibrocytes, World Scientc Pub Co Inc: 1-18
Bucala R, Spiegel L A, Chesney J, Hogan M, Cerami A (1994) Circulating fibrocytes define a new leukocyte subpopulation that mediates tissue repair. Mol Med 1: 71-81
Chung Y C, Chang K J, Yang C C, Lai M T, Hsu C P, Hsuch S F, Peng C C, Fu H H, Chang Y F, Yang S D (2002) Association of proline-directed protein kinase FA with tumorigenesis, invasion, and poor prognosis of human colon carcinoma. Cancer 95: 1840-1847
Condeelis J, Pollard J W (2006) Macrophages: obligate partners for tumor cell migration, invasion, and metastasis. Cell 124: 263-266
De Wever O, Mareel M (2003) Role of tissue stroma in cancer cell invasion. J Pathol 200: 429-447
Direkze N C, Alison M R (2006) Bone marrow and tumour stroma: an intimate relationship. Hematol Oncol 24: 189-195
Dunsmore S E, Shapiro S D (2004) The bone marrow leaves its scar: new concepts in pulmonary fibrosis. J Clin Invest 113: 180-182
Fu H H, Yang S D (2004) Suppression of proline-directed protein kinase F(A) inhibits the malignant growth of human pancreatic ductal adenocarcinoma. Anticancer Res 24: 1489-1494
Grivennikov S I, Greten F R, Karin M (2010) Immunity, inflammation, and cancer. Cell 140: 883-899
Hanahan D, Weinberg R A (2011) Hallmarks of cancer: the next generation. Cell 144: 646-674
Hsu C P, Hsuch S F, Yang C C, Yang S D (2000) Suppression of proline-directed protein kinase F(A) expression inhibits the growth of human chronic myeloid leukaemia cells. Br J Cancer 82: 1480-1484
Hsu C P, Yang C C, Hsuch S F, Peng C C, Fu H H, Yang S D (2001) Suppression of proline-directed protein kinase F(A) potentiates apoptotic induction and greatly enhances chemosensitivity in human acute lymphoblastic leukemia cells. Cancer 92: 1753-1758
Hsu C P, Yang C C, Yang S D (2000) Suppression of proline-directed protein kinase F(A) expression potentiates erythroid differentiation of human myeloid leukemia cells. Cancer 89: 1004-1011
Hsu C P, Yang C C, Yang S D (2001) Suppression of proline-directed protein kinase F(A) systemically inhibits the growth of human acute leukemia cells. Int J Cancer 91: 650-653
Hsuch S F, Lai M T, Yang C C, Chung Y C, Hsu C P, Peng C C, Fu H H, Cheng Y M, Chang K J, Yang S D (2002) Association of overexpressed proline-directed protein kinase F(A) with chemoresistance, invasion, and recurrence in patients with bladder carcinoma. Cancer 95: 775-783
Karnoub A E, Dash A B, Vo A P, Sullivan A, Brooks M W, Bell G W, Richardson A L, Polyak K, Tubo R, Weinberg R A (2007) Mesenchymal stem cells within tumour stroma promote breast cancer metastasis. Nature 449: 557-563
Kisseleva T, Brenner D A (2008) Mechanisms of fibrogenesis. Exp Biol Med 233: 109-122
Le Bousse-Kerdiles M C, Martyre M C, Samson M (2008) Cellular and molecular mechanisms underlying bone marrow and liver fibrosis: a review. Eur Cytokine Netw 19: 69-80
Lin W R, Brittan M, Alison M R (2008) The role of bone marrow-derived cells in fibrosis. Cells Tissues Organs 188: 178-188
Mishra P J, Glod J W, Banerjee D (2009) Mesenchymal stem cells: flip side of the coin. Cancer Res 69: 1255-1258
Moioli E K, Clark P A, Chen M, Dennis J E, Erickson H P, Gerson S L, Mao J J (2008) Synergistic actions of hematopoietic and mesenchymal stem/progenitor cells in vascularizing bioengineered tissues. PLoS ONE 3: e3922
Schafer M, Werner S (2008) Cancer as an overhealing wound: an old hypothesis revisited. Nat Rev Mol Cell Biol 9: 628-638
Schmidt M, Sun G, Stacey M A, Mori L, Mattoli S (2003) Identification of circulating fibrocytes as precursors of bronchial myofibroblasts in asthma. J Immunol 171: 380-389
Valtieri M, Sorrentino A (2008) The mesenchymal stromal cell contribution to homeostasis. J Cell Physiol 217: 296-300
Vandenheede J R, Yang S D, Goris J, Merlevede W (1980) ATP Mg-dependent protein phosphatase from rabbit skeletal muscle. II. Purification of the activating factor and its characterization as a bifunctional protein also displaying synthase kinase activity. J Biol Chem 255: 11768-11774
Woodgett J R (1990) Molecular cloning and expression of glycogen synthase kinase-3/factor A. EMKO J9: 2431-2438
Wynn T A (2008) Cellular and molecular mechanisms of fibrosis. J Pathol 214: 199-210
Yang C C, Hsu C P, Yang S D (2000) Antisense suppression of proline-directed protein kinase FA enhances chemosensitivity in human prostate cancer cells. Clin Cancer Res 6: 1024-1030
Yang S D (2004) Proline-directed protein kinase F_{A} as a potential target for diagnosis and therapy of human cancers. Curr Cancer Drug Targets 4: 591-596
Yang S D, Vandenheede J R, Goris J, Merlevede W (1980) ATP x Mg-dependent protein phosphatase from rabbit skeletal muscle. I. Purification of the enzyme and its regulation by the interaction with an activating protein factor. J Biol Chem 255: 11759-11767
Yin T, Li L (2006) The stem cell niches in bone. J Clin Invest 116: 1195-1201

What is claimed is:

1. A method for detecting the presence of a cellular expression profile in a subject indicative of unhealthy bone marrow-derived cell (BMDC), which method comprises:
obtaining a biological sample from said subject;
determining a bone marrow-derived cell (BMDC) in the biological sample; and
determining the expression of PDPK F_{A}/GSK-3α in the BMDC;
wherein an aberrant intracellular accumulation of PDPK F_{A}/GSK-3α in BMDC in comparison to a normal cell indicates the subject has the presence of unhealthy BMDC.

2. The method of claim 1, wherein said biological sample is selected from the group consisting of bone marrow, cord blood, peripheral blood, tissue sample, ascites, pleural effusions and body fluids.

3. The method of claim 1, wherein said expression of PDPK F_{A}/GSK-3α is determined by assessing PDPK F_{A}/GSK-3α protein level.

4. The method of claim 1, wherein BMDC is a stem/progenitor cell or derivative, selected from a group consisting of bone marrow-derived cell, mesenchymal stem/progenitor cell, hematopoietic stem/progenitor cell, fibrocyte, macrophage, fibroblast, myofibroblast and mesenchymal cell.

5. The method of claim 1, wherein the aberrant intracellular accumulation of PDPK FA/GSK-3α is in cytoplasmic or nuclear expression level.

6. A method for detecting the presence of a cellular expression profile in a subject indicative of unhealthy bone marrow-derived cell (BMDC) for disease predisposition, which method comprises:
obtaining a biological sample from said subject;
determining a bone marrow-derived cell (BMDC) in the biological sample; and
determining the expression of PDPK F_{A}/GSK-3α in the BMDC;
wherein an aberrant intracellular accumulation of PDPK F_{A}/GSK-3α in BMDC in comparison to a normal cell indicates the subject has the presence of unhealthy BMDC.

7. The method of claim 6 wherein said biological sample is selected from the group consisting of bone marrow, cord blood, peripheral blood, tissue sample, ascites, pleural effusions and body fluids.

8. The method of claim 6 wherein said expression of PDPK F_{A}/GSK-3α is determined by assessing PDPK F_{A}/GSK-3α protein level.

9. The method of claim 6 wherein BMDC is a stem/progenitor cell or derivative, selected from a group consisting of bone marrow-derived cell, mesenchymal stem/progenitor cell, hematopoietic stem/progenitor cell, fibrocyte, macrophage, fibroblast, myofibroblast and mesenchymal cell.

10. The method of claim 6 wherein the aberrant intracellular accumulation of PDPK FA/GSK-3α is in cytoplasmic or nuclear expression level.

11. The method of claim 6 wherein said disease predisposition is selected from a group consisting of very early-stage aberrant wound healing, chronic inflammation and immune disorder towards the end-stage fibrotic disease and neoplasia.

12. The method of claim 6 wherein the subject is free of diagnosed cancer defined by the current clinic cancer definitions and parameters applicable worldwide.

13. A kit for detection of unhealthy bone marrow-derived cell (BMDC) in a biological sample, comprising:
instructions to obtain a biologic sample from a subject;
instructions to determine a bone marrow-derived cell (BMDC) in the biological sample;
instructions to determine the expression profile of PDPK FA/GSK-3α in said bone marrow-derived cell (BMDC), and
specific antibody and reagents to assessing PDPK FA/GSK-3α protein expressions pattern for determining the presence of an unhealthy BMDC.
